(19) United States
(12) Patent Application Publication
(10) Pub. No.: US 2014/0222474 A1
(43) Pub. Date: Aug. 7, 2014

(54) SYSTEM AND METHOD FOR CERTIFYING ATTENDANCE AT A PROMOTIONAL EVENT

(71) Applicant: HSC Acquisition, LLC dba HealthSTAR Communications, (US)

(72) Inventor: Al Mahafzah, Wayne, NJ (US)

(73) Assignee: HSC Acquisition, LLC dba HealthSTAR Communications, Mahwah, NJ (US)

(21) Appl. No.: 13/758,545

(22) Filed: Feb. 4, 2013

(51) Int. Cl. G06Q 10/02; G06Q 50/22

(52) U.S. Cl. CPC G06Q 10/02; G06Q 50/22
USPC 705/5

(57) ABSTRACT

In a method for certifying attendance at a healthcare promotional event a computer retrieves data indicative of a healthcare professional's pre-registration for a healthcare promotional event. A computer prompts for a signature, wherein the signature confirms the healthcare professional's attendance at the healthcare promotional event. A computer receives data representative of the signature. A computer prompts for a response to a presented certification statement. A computer certifies the healthcare professional's attendance at the healthcare promotional event responsive to receiving a response to the certification statement and responsive to receiving the data representative of the signature.

### FIELD OF INVENTION

The present disclosure relates to the field of promotional events. More particularly, the present invention relates to a system and method for certifying attendance at a promotional event.

### BACKGROUND

Industry opinion leaders are individuals considered to be effective at influencing others within an industry to make certain choices and to conform their opinions to be consistent with those of the opinion leader. In the healthcare industry specifically, a key opinion leader (KOL) is a physician who is capable of influencing other physicians to prescribe a certain drug produced by a specific drug manufacturer. Thus, it is desirable for a healthcare marketing company that is promoting a specific drug, to arrange a promotional event, such as a dinner party, in which a KOL is able to share his opinion regarding the drug with other healthcare professionals.

Healthcare promotional events, however, are regulated and require healthcare marketing companies and drug companies to comply with specific rules. The rules include certifying attendance at a promotional event which includes both confirming attendance as well as confirming specific conditions or requirements of the attendees. Certifying attendance manually using traditional paper records, however, may be tedious and time consuming, as well as error prone.

### SUMMARY OF THE INVENTION

In a method for certifying attendance at a healthcare promotional event a computer retrieves data indicative of a healthcare professional's pre-registration for a healthcare promotional event. A computer prompts for a signature, wherein the signature confirms the healthcare professional's attendance at the healthcare promotional event. A computer receives data representative of the signature. A computer prompts for a response to a presented certification statement. A computer certifies the healthcare professional's attendance at the healthcare promotional event responsive to receiving a response to the certification statement and responsive to receiving the data representative of the signature.

A system for certifying attendance at a healthcare promotional event comprises at least one processor, at least one computer-readable tangible storage device, and program instructions stored on the at least one storage device for execution by the at least one processor. The program instructions comprise first program instructions configured to retrieve data indicative of a healthcare professional's pre-registration for a healthcare promotional event. The program instructions further comprise second program instructions configured to prompt for a signature, wherein the signature confirms the healthcare professional's attendance at the healthcare promotional event. The program instructions further comprise third program instructions configured to receive data representative of the signature. The program instructions further comprise fourth program instructions configured to prompt for a response to a presented certification statement. The program instructions further comprise fifth program instructions configured to certify the healthcare professional's attendance at the healthcare promotional event responsive to receiving a response to the certification statement and responsive to receiving the data representative of the signature.

A computer program product for facilitating a virtual promotional event comprising at least one computer-readable tangible storage device and program instructions stored on the at least one storage device. The program instructions comprise first program instructions configured to retrieve data indicative of a healthcare professional's pre-registration for a healthcare promotional event. The program instructions further comprise second program instructions configured to prompt for a signature, wherein the signature confirms the healthcare professional's attendance at the healthcare promotional event. The program instructions further comprise third program instructions configured to receive data representative of the signature. The program instructions further comprise fourth program instructions configured to prompt for a response to a presented certification statement. The program instructions further comprise fifth program instructions configured to certify the healthcare professional's attendance at the healthcare promotional event responsive to receiving a response to the certification statement and responsive to receiving the data representative of the signature.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example system for certifying attendance at a healthcare promotional event.

FIG. 2 illustrates a block diagram of an example computer of FIG. 1 for certifying attendance at a healthcare promotional event.

FIG. 3 is a flow chart illustrating an example method for certifying attendance at a healthcare promotional event.

FIG. 4 is a schematic diagram of an example computer of FIG. 1 for certifying attendance at a healthcare promotional event.

### DETAILED DESCRIPTION

The following includes definitions of selected terms employed herein. The definitions include various examples, forms, or both of components that fall within the scope of a term and that may be used for implementation. The examples are not intended to be limiting. Both singular and plural forms of terms may be within the definitions.

“Computer communication,” as used herein, refers to a communication between two or more computing devices (e.g., computer, personal digital assistant, cellular telephone) and can be, for example, a network transfer, a file transfer, an applet transfer, an email, a hypertext transfer protocol (HTTP) transfer, and so on. A computer communication can occur across, for example, a wireless system (e.g., IEEE 802.11, IEEE 802.15), an Ethernet system (e.g., IEEE 802.3), a token ring system (e.g., IEEE 802.5), a local area network (LAN), a wide area network (WAN), a point-to-point system, a circuit switching system, a packet switching system, combinations thereof, and so on.

“Computer-readable medium,” as used herein, refers to a medium that participates in directly or indirectly providing signals, instructions, or data. A computer-readable medium may take forms, including, but not limited to, non-volatile media, volatile media, and transmission media. Non-volatile media may include, for example, optical or magnetic disks, and so on. Volatile media may include, for example, optical or magnetic disks, dynamic memory, and the like. Transmission media may include coaxial cables, copper wire, fiber optic cables, and the like. Transmission media can also take the form of electromagnetic radiation, like that generated during radio-wave and infra-red data communications, or take the form of one or more groups of signals. Common forms of a computer-readable medium include, but are not limited to, a floppy disk, a flexible disk, a hard disk, a magnetic tape, other magnetic media, a CD-ROM, other optical media, punch cards, paper tape, other physical media with patterns of holes, a RAM, a ROM, an EPROM, a FLASH-EPROM, or other memory chip or card, a memory stick, a carrier wave/pulse, Phase Change Memory, and other media from which a computer, a processor, or other electronic device can read. Signals used to propagate instructions or other software over a network, like the Internet, can be considered a “computer-readable medium.”

“Data store,” as used herein, refers to a physical or logical entity that can store data. A data store may be, for example, a database, a table, a file, a list, a queue, a heap, a memory, a register, and so on. A data store may reside in one logical or physical entity or may be distributed between two or more logical or physical entities.

“Logic,” as used herein, includes but is not limited to hardware, firmware, software, or combinations of each to perform a function(s) or an action(s), or to cause a function or action from another logic, method, or system. For example, based on a desired application or needs, logic may include a software controlled microprocessor, discrete logic like an application specific integrated circuit (ASIC), a programmed logic device, a memory device containing instructions, or the like. Logic may include one or more gates, combinations of gates, or other circuit components. Logic may also be fully embodied as software. Where multiple logical logics are described, it may be possible to incorporate the multiple logical logics into one physical logic. Similarly, where a single logical logic is described, it may be possible to distribute that single logical logic between multiple physical logics.

An “operable connection,” or a connection by which entities are “operably connected,” is one in which signals, physical communications, or logical communications may be sent or received. Typically, an operable connection includes a physical interface, an electrical interface, or a data interface, but it is to be noted that an operable connection may include differing combinations of these or other types of connections sufficient to allow operable control. For example, two entities can be operably connected by being able to communicate signals to each other directly or through one or more intermediate entities like a processor, operating system, a logic, software, or other entity. Logical or physical communication channels can be used to create an operable connection.

“Software,” as used herein, includes but is not limited to, one or more computer or processor instructions that can be read, interpreted, compiled, or executed and that cause a computer, processor, or other electronic device to perform functions, actions, or behave in a desired manner. The instructions may be embodied in various forms like routines, algorithms, modules, methods, threads, or programs including separate applications or code from dynamically or statically linked libraries. Software may also be implemented in a variety of executable or loadable forms including, but not limited to, a stand-alone program, a function call (local or remote), a servelet, an applet, instructions stored in a memory, part of an operating system, or other types of executable instructions. It will be appreciated by one of ordinary skill in the art that the form of software may depend, for example, on requirements of a desired application, the environment in which it runs, or the desires of a designer/programmer or the like. It will also be appreciated that computer-readable or executable instructions can be located in one logic or distributed between two or more communicating, co-operating, or parallel processing logics and thus can be loaded or executed in serial, parallel, massively parallel, and other manners.

Suitable software for implementing the various components of the example systems and methods described herein may be produced using programming languages and tools like Java, Java Script, Java.NET, ASP.NET, VB.NET, Cocoa, Pascal, C#, C++, C, CGI, Perl, SQL, APIs, SDKs, assembly, firmware, microcode, or other languages and tools. Software, whether an entire system or a component of a system, may be embodied as an article of manufacture and maintained or provided as part of a computer-readable medium as defined previously. Another form of the software may include signals that transmit program code of the software to a recipient over a network or other communication medium. Thus, in one example, a computer-readable medium has a form of signals that represent the software/firmware as it is downloaded from a web server to a user. In another example, the computer-readable medium has a form of the software/firmware as it is maintained on the web server. Other forms may also be used.

“User,” as used herein, includes but is not limited to one or more persons, software, computers or other devices, or combinations of these.

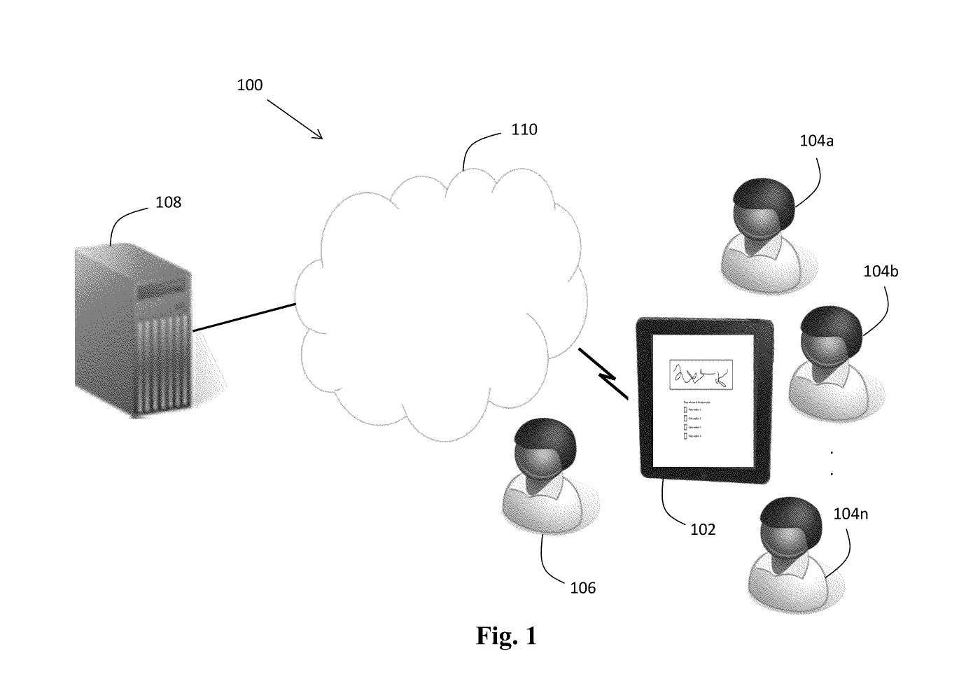

FIG. 1 illustrates an example system 100 for certifying attendance at a promotional event. Although the example system and method described herein makes reference to healthcare promotional events, it should be understood that the example system and method may similarly be used to certify attendance at any other suitable event. For example, the system and method may be used to certify attendance at an exam, at a training session, at a meeting, and so on. Similarly, although the example system and method makes reference to healthcare professionals, it should be understood that the example system and method may be used to certify attendance of other suitable attendees as well.

System 100 includes a computer 102 configured to interface with one or more healthcare professionals 104a, 104b, and 104n (hereinafter referred to as healthcare professional 104) and to certify that healthcare professional 104 attended a healthcare promotional event. It should be understood that, although computer 102 is illustrated as a portable tablet computer, computer 102 may also include any type of similar computing device capable of interfacing with healthcare professional 104 and certifying that healthcare professional 104 attended a healthcare promotional event. For example, computer 102 may include a mobile phone such as a smartphone, a laptop computer, a desktop computer, and so on.

Computer 102 is made available to healthcare professional 104, at a healthcare promotional event, by a healthcare promotional event organizer 106 (hereinafter referred to as organizer) in order to certify healthcare professional's 104 attendance at the healthcare promotional event. Computer 102 is configured to retrieve, from a data server 108 via Internet 110, a list of healthcare professionals that have been pre-registered for the healthcare promotional event and to provide organizer 106 with the list. The list may include names of healthcare professionals as well as other suitable information such as contact information, date registered, area of specialty, hospital affiliation, and so on.

The organizer 106 presents the computer to each pre-registered healthcare professional 104 in order to obtain a digital signature via an interface of computer 102. For example, computer 102 may be configured to capture a digital signature of healthcare professional 104 by receiving input from a stylus via a touch screen interface. Receipt of the digital signature confirms that healthcare professional 104 attended the healthcare promotional event.

In addition, computer 102 is configured to prompt healthcare professional 104 for a response to one or more certification statements. Certification statements are predefined in order to illicit information necessary to ensure compliance with industry regulations. For example, industry regulations may prohibit a government employee to consume a meal while attending a healthcare promotional event. Thus, in addition to requiring healthcare professional 104 to provide a signature, computer 102 may ask healthcare professional 104 to certify that he/she had not consumed a meal during the healthcare promotional event.

Certification questions may also be predefined by organizer 106 in order to illicit information specific to the event. For example, organizer 106 may wish to limit attendance at a particular event to non-Government employees. Accordingly, computer 102 may ask healthcare professional 104 to certify that he/she is not a government employee.

Computer 102 is configured to prompt healthcare professional 104 for a response to a certification question by presenting a question or a statement via a display screen or via another suitable interface. Questions or statements, and corresponding options for responding, may be presented using checkboxes, buttons, radio buttons, audio or video recordings, or in other suitable form.

Computer 102 is further configured to certify that healthcare professional 104 attended the healthcare promotional event by associating a signature of the healthcare professional with the responses to the certification statements or questions (hereinafter referred to as certification data). Computer 102 may either store certification data locally or wirelessly transfer certification data to data server 108 via Internet 110.

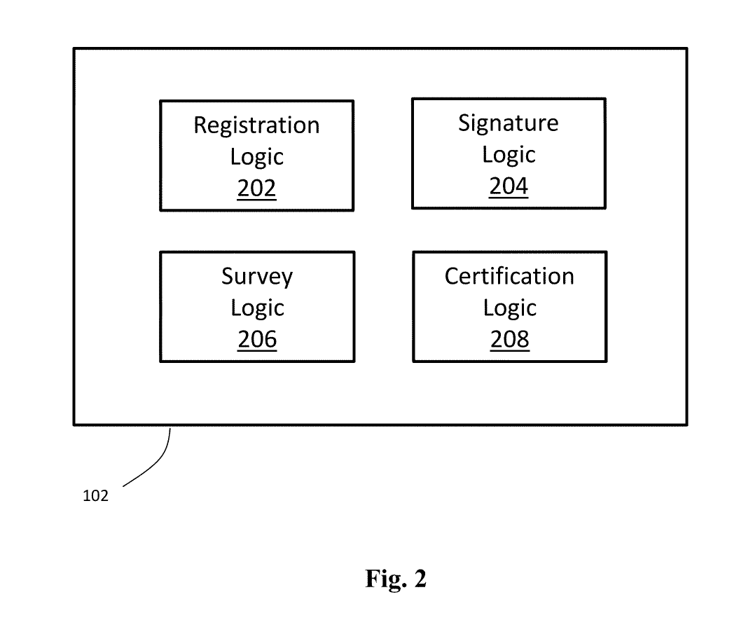

FIG. 2 illustrates a block diagram of an example computer 102 of FIG. 1 for certifying attendance at a promotional event. Computer 102 includes registration logic 202 configured to retrieve healthcare professional's 104 pre-registration data for a healthcare promotional event and to present the data to organizer 106. In one example, registration logic 202 may be configured to require organizer 106 to provide login credentials. Accordingly, registration logic 202 may be configured to receive login credentials and to enable organizer 106 to access pre-registration data for a healthcare promotional event based on the login credentials. For example, if a first organizer organizes a first group of healthcare professionals for a healthcare promotional event and a second organizer organizes a second group of healthcare professionals for the same healthcare promotional event or for a different healthcare promotional event, registration logic 202 is configured to allow the first organizer to only access pre-registration data related to, and in turn certify attendance of, the first group of healthcare professionals.

In one example, registration logic 202 may be configured to enable organizer 106 to modify information associated with pre-registered healthcare professionals, during a healthcare promotional event or at the conclusion of a healthcare promotional event. Specifically, registration logic 202 is configured to receive a request to modify information associated with healthcare professional's 104 pre-registration for a healthcare promotional event and to modify the pre-registration accordingly. For example, registration logic 202 may enable organizer 106 to update contact information such as an email address or a phone number of healthcare professional 104. In one example, registration logic 202 enables healthcare professional 104 to update contact information as well.

In one example, registration logic 202 may be configured to enable organizer 106 to add additional healthcare professionals to a list of pre-registered healthcare professionals. Specifically, registration logic 202 is configured to receive a request to register a second healthcare professional for a healthcare promotional event and to modify the registration information for the healthcare promotional event accordingly. Thus, organizer 106 may include healthcare professionals that were not pre-registered for a healthcare promotional event, in the final certification of attendees of the event.

Computer 102 further includes signature logic 204 configured to prompt for and receive a digital image of a signature from healthcare professional 104. Signature logic 204 may receive the signature from a stylus or from a user's finger via a touch screen interface or via another suitable interface. In one example, signature logic 204 is configured to convert the image of the signature to binary code to reduce memory requirements for storing the signature and to reduce network bandwidth requirements for transmitting the signature.

Computer 102 further includes survey logic 102 configured to present one or more certification statements or questions to healthcare professional 104 and to prompt for a response. In one example, survey logic 206 may be configured to enable organizer 106 to create certification questions on the fly, during a healthcare promotional event or at the conclusion of the healthcare promotional event. This enables organizer 106 to certify certain situations or conditions that organizer 106 may not have originally anticipated or planned for.

Computer 102 further includes certification logic 208 configured to certify healthcare professional's 104 attendance at a healthcare promotional event after receiving a response to one or more certification statements and after receiving a signature. Certification logic 208 may certify attendance by storing responses to certification statements in association with a signature (hereinafter referred to as certification data), by transmitting certification data to data server 108, by emailing certification data to healthcare professional 104, or by storing or transmitting the certification data in another suitable manner.

In one example, certification logic 208 is configured to close the healthcare promotional event and prevent additional healthcare professionals from being certified as being in attendance after the healthcare promotional event is closed. Closing a healthcare promotional event may include locking data associated with the healthcare promotional event or other suitable actions to prevent inclusion of additional healthcare professionals in the healthcare promotional event.

In one example, registration logic 202 is configured to retrieve pre-registration data of a healthcare promotional event for a plurality of healthcare professionals. In such an example, certification logic 208 may be configured to certify the plurality of healthcare professionals' attendance at the healthcare promotional event after receiving responses to the certification statements from all of healthcare professionals and after receiving signatures from all of the healthcare professionals.

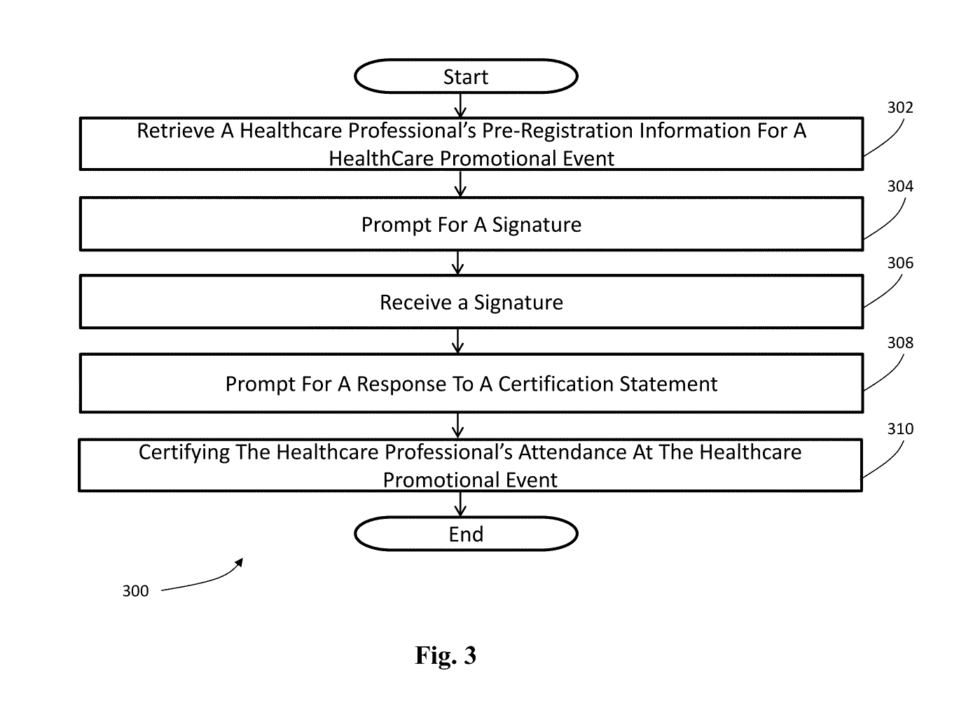

FIG. 3 is a flow chart illustrating an example method for certifying attendance at a healthcare promotional event. At step 302, computer 102 retrieves healthcare professional's 104 pre-registration information for a healthcare promotional event. At step 304, computer 102 prompts healthcare professional 104 for a signature. At step 306, computer 102 receives a signature from healthcare professional, in the form of an electronic image, via a suitable image capture interface.

At step 308, computer 102 prompts healthcare professional 104 for a response to a certification statement. At step 310, computer 102 certifies healthcare professional's 104 attendance at a healthcare promotional event after receiving a response to the certification statement and after receiving the signature. In one example, computer 102 transmits the response to the certification statement in association with the signature to data server 108.

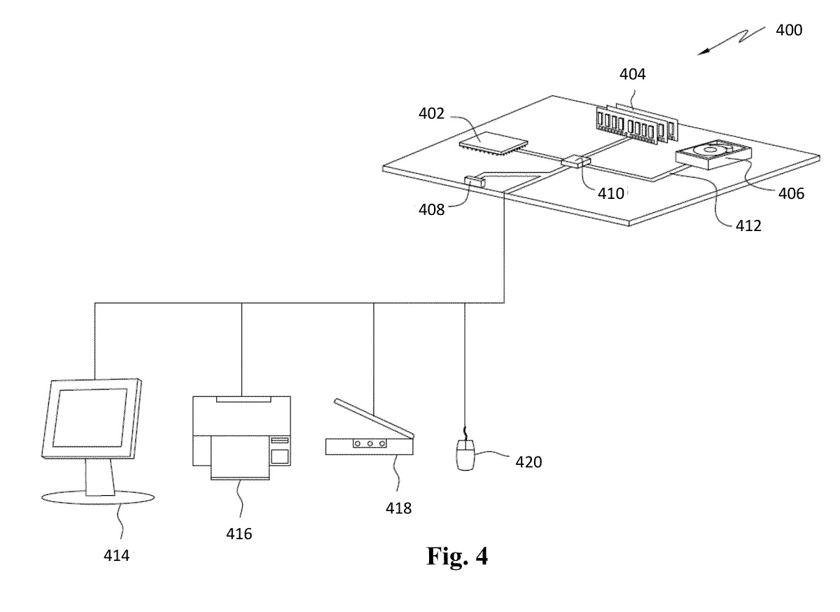

FIG. 4 is a block diagram of an example computer 400 for implementing the system and method for certifying attendance at a promotional event. The example computer 400 is intended to represent various forms of digital computers, including laptops, desktops, handheld computers, tablet computers, servers, and other similar types of computing devices. Computer 400 includes a processor 402, memory 404, a storage device 406, and a communication port 408, operably connected by an interface 410 via a bus 412.

Storage device 406 can store registration logic 202, signature logic 204, survey logic 206, and certification logic 208.

Processor 402 processes instructions, via memory 404, for execution within computer 400. In an example embodiment, multiple processors along with multiple memories may be used.

Memory 404 may be volatile memory or non-volatile memory. Memory 404 may be a computer-readable medium, such as a magnetic disk or optical disk. Storage device 406 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 404 or storage device 406. The computer program product may contain registration logic 202, signature logic 204, survey logic 206, and certification logic 208.

Computer 400 can be coupled to one or more input and output devices such as a display 414, a printer 416, a scanner 418, and a mouse 420.

While example systems, methods, and so on, have been illustrated by describing examples, and while the examples have been described in considerable detail, it is not the intention to restrict or in any way limit the scope of the appended claims to such detail. It is, of course, not possible to describe every conceivable combination of components or methodologies for purposes of describing the systems, methods, and so on, described herein. Additional advantages and modifications will readily appear to those skilled in the art. Therefore, the invention is not limited to the specific details, and illustrative examples shown or described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

Some portions of the detailed descriptions are presented in terms of algorithms and symbolic representations of operations on data bits within a memory. These algorithmic descriptions and representations are the means used by those skilled in the art to convey the substance of their work to others. An algorithm is here, and generally, conceived to be a sequence of operations that produce a result. The operations may include physical manipulations of physical quantities. Usually, though not necessarily, the physical quantities take the form of electrical or magnetic signals capable of being stored, transferred, combined, compared, and otherwise manipulated in a logic and the like.

1. A method for certifying attendance at a healthcare promotional event, the method comprising the steps of:
a computer retrieving data indicative of a healthcare professional's pre-registration for a healthcare promotional event;
a computer prompting for a signature, wherein the signature confirms the healthcare professional's attendance at the healthcare promotional event;
a computer receiving data representative of the signature;
a computer prompting for a response to a presented certification statement; and
a computer certifying the healthcare professional's attendance at the healthcare promotional event responsive to receiving a response to the certification statement and responsive to receiving the data representative of the signature.

2. The method of claim 1, further comprising the steps of:
a computer receiving login credentials; and
a computer enabling access to the data indicative of a healthcare professional's pre-registration for a healthcare promotional event based on the login credentials.

3. The method of claim 1, wherein the step of the computer certifying the healthcare professional's attendance comprises transmitting data representative of the response to the certification statement in association with the data representative of the signature.

4. The method of claim 1, further comprising the step of a computer converting the data representative of the image to binary code.

5. The method of claim 1, further comprising the steps of:
a computer receiving data representative of a request to modify information associated with the healthcare professional's pre-registration for the healthcare promotional event; and
a computer modifying registration information associated with the promotional event to include the requested modification.

6. The method of claim 1, further comprising the steps of:
a computer receiving data representative of a request to register a second healthcare professional for the healthcare promotional event; and
a computer modifying registration information associated with the healthcare promotional event to include registration information for the second healthcare professional.

7. The method of claim 1, further comprising the steps of:
a computer retrieving data indicative of a plurality of healthcare professionals' pre-registration for a healthcare promotional event; and
a computer certifying the healthcare professionals' attendance at the healthcare promotional event responsive to receiving a response to the certification statement from all of the plurality of healthcare professionals and responsive to receiving data representative of a signature from all of the plurality of healthcare professionals.

8. A system for certifying attendance at a healthcare promotional event, the system comprising:
at least one processor, at least one computer-readable tangible storage device, and program instructions stored on the at least one storage device for execution by the at least one processor, the program instructions comprising:
first program instructions configured to retrieve data indicative of a healthcare professional's pre-registration for a healthcare promotional event;
second program instructions configured to prompt for a signature, wherein the signature confirms the healthcare professional's attendance at the healthcare promotional event;
third program instructions configured to receive data representative of the signature;
fourth program instructions configured to prompt for a response to a presented certification statement; and
fifth program instructions configured to certify the healthcare professional's attendance at the healthcare promotional event responsive to receiving a response to the certification statement and responsive to receiving the data representative of the signature.

9. The system of claim 8, the program instructions further comprising:
sixth program instructions configured to receive login credentials; and
seventh program instructions configured to enable access to the data indicative of a healthcare professional's pre-registration for a healthcare promotional event based on the login credentials.

10. The system of claim 8, wherein the fifth program instructions are further configured to transmit data representative of the response to the certification statement in association with the data representative of the signature.

11. The system of claim 8, the program instructions further comprising sixth program instructions configured to convert the data representative of the image to binary code.

12. The system of claim 8, the program instructions further comprising:
sixth program instructions configured to receive data representative of a request to modify information associated with the healthcare professional's pre-registration for the healthcare promotional event; and
seventh program instructions configured to modify registration information associated with the promotional event to include the requested modification.

13. The system of claim 8, the program instructions further comprising:
sixth program instructions configured to receive data representative of a request to register a second healthcare professional for the healthcare promotional event; and
seventh program instructions configured to modify registration information associated with the healthcare promotional event to include registration information for the second healthcare professional.

14. The system of claim 8, the program instructions further comprising:
sixth program instructions configured to retrieve data indicative of a plurality of healthcare professionals' pre-registration for a healthcare promotional event; and
seventh program instructions configured to certify the healthcare professionals' attendance at the healthcare promotional event responsive to receiving a response to the certification statement from all of the plurality of healthcare professionals and responsive to receiving data representative of a signature from all of the plurality of healthcare professionals.

15. A computer program product for facilitating a virtual promotional event, the computer program product comprising:
at least one computer-readable tangible storage device and program instructions stored on the at least one storage device, the program instructions comprising:
first program instructions configured to retrieve data indicative of a healthcare professional's pre-registration for a healthcare promotional event;
second program instructions configured to prompt for a signature, wherein the signature confirms the healthcare professional's attendance at the healthcare promotional event;
third program instructions configured to receive data representative of the signature;
fourth program instructions configured to prompt for a response to a presented certification statement; and
fifth program instructions configured to certify the healthcare professional's attendance at the healthcare promotional event responsive to receiving a response to the certification statement and responsive to receiving the data representative of the signature.

16. The computer program product of claim 15, the program instructions further comprising:
sixth program instructions configured to receive login credentials; and
seventh program instructions configured to enable access to the data indicative of a healthcare professional's pre-registration for a healthcare promotional event based on the login credentials.

17. The computer program product of claim 15, wherein the fifth program instructions are further configured to transmit data representative of the response to the certification statement in association with the data representative of the signature.

18. The computer program product of claim 15, the program instructions further comprising sixth program instructions configured to convert the data representative of the image to binary code.

19. The computer program product of claim 15, the program instructions further comprising:
sixth program instructions configured to receive data representative of a request to modify information associated with the healthcare professional's pre-registration for the healthcare promotional event; and
seventh program instructions configured to modify registration information associated with the promotional event to include the requested modification.

20. The computer program product of claim 15, the program instructions further comprising:
sixth program instructions configured to receive data representative of a request to register a second healthcare professional for the healthcare promotional event; and
seventh program instructions configured to modify registration information associated with the healthcare promotional event to include registration information for the second healthcare professional.
